(19) United States
(12) Patent Application Publication
(10) Pub. No.: US 2007/0282628 A1
(43) Pub. Date: Dec. 6, 2007

(54) Method and Apparatus for Managing Rejections and Denials of Payments for Medical Services

(76) Inventors: Elizabet Satterfield, Seattle, WA (US); Michael Garrett, Anacortes, WA (US); Eileen Schmidt, Kalispell, MT (US); Susan Cohen, Chestnut Hill, MA (US)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 11/419,125

(22) Filed: May 18, 2006

(51) Int. Cl. G06Q 10/00

(52) U.S. Cl. 705/2

(57) ABSTRACT

A payment rejection and denial management system receives a remit in response to a claim submitted for rendered medical services, processes the remit, and executes automatic actions to manage claim rejections and denials. Such a system allows medical service providers to recover payments incorrectly rejected or denied and to seek payments from secondary payers or the patient themselves.

### BACKGROUND OF THE INVENTION

1. Field of the Invention

This invention relates in general to managing patient accounts in a healthcare system.

2. Description of the Related Art

Maintaining healthy cash flow on a monthly basis is essential for medical service providers as the planning and budgeting of the business is done based on the revenue generated by accounts receivables. Identifying uncollected claims and turning them into cash is very important for businesses.

Currently, when a medical service provider submits a claim for medical services to a payer, the payer often does not pay the entire bill and instead either declines paying the claim or provides a partial payment. When payers do not pay the entire bill, they provide codes that indicate reasons for the lack of payment. These reasons fall into various categories, such as coding errors, a problem with insurance eligibility, etc. Medical service providers use these codes to identify and rectify errors in order to obtain maximum payments. As a result, the medical staff attempts to rectify errors and re-submit the claims in the hope that the claim will be paid. The process of collection takes a tremendous amount of employees' time to collect on the claims. Many of the rejected claims get lost in the shuffle and are never collected.

What is needed is a mechanism for efficiently managing rejections and denials of medical claims so that medical service providers can recover payments incorrectly rejected or denied in error and reduce the time it takes to seek payments from secondary payers or patients.

### SUMMARY OF THE INVENTION

The above need is met by a payment rejection and denial management system that receives a remit in response to a claim submitted for rendered medical services, processes the remit, and executes automatic actions aimed at collecting payments for rendered medical services. Such a system allows medical service providers to recover payments incorrectly rejected or denied, thereby maintaining healthy revenue streams.

In one embodiment, the payment rejection and denial management system executes various engines to perform the required functionality. These engines include a remit processing engine, a rejection and denial engine, an event engine, a billing engine, a worklist engine, and a tickler engine.

In one aspect, the rejection and denial engine allows a user to create profile entries using a reason and remark profile engine that will determine the automatic actions according to various attributes of the incoming payments. A profile entry has a rule set associated with one or more actions. A rule set includes a group of payment attributes and values selected by a user. A user can provide values that indicate the auctions to be performed. Once the system selects the appropriate rule set(s), it processes the values for the associated action(s). The rejection and denial engine processes customer definable settings that will result in ‘no match’ or ‘best match’ on a rule set defined for the reason and remark profile engine. If there is no match, payment processing continues. If a ‘best match’ is made, the system automatically begins processing one or more actions.

In one embodiment, the billing engine submits a claim to a third party system for rendered medical services. The remit processing engine receives a remit indicating how the claim was adjudicated, processes the remit to validate data on the remit, and submits validated data to the rejection and denial engine. The validated data includes values for various attributes on the remit. The rejection and denial engine uses the reason and remark profile engine to identify one or more profile entries that match the received data. The rejection and denial engine identifies the “best match”—a profile entry that matches on the greatest number of attributes. The rejection and denial engine selects one or more actions associated with the best match and executes one or more automatic actions.

The features and advantages described in the specification are not all inclusive and, in particular, many additional features and advantages will be apparent to one of ordinary skill in the art in view of the drawings, specification, and claims. Moreover, it should be noted that the language used in the specification has been principally selected for readability and instructional purposes, and may not have been selected to delineate or circumscribe the inventive subject matter.

### BRIEF DESCRIPTION OF THE DRAWINGS

FIG. 1 is a high-level diagram illustrating the environment in which the present invention operates.

FIG. 2 is a block diagram of the components of a payment rejection and denial management system.

FIG. 3 is a block diagram of the components of a rejection and denial engine executed by the payment rejection and denial management system.

FIG. 4 is an exemplary user interface provided by a reason and remark profile engine.

FIG. 5 is an exemplary reason and remark action profile user interface.

FIG. 6 is an event diagram of a method performed by the payment rejection denial and management system.

FIG. 7 is a flow diagram of a method performed by a tickler engine.

FIG. 8 is a flow diagram of a method performed by a worklist engine.

FIG. 9 is a flow diagram of a method performed by a billing engine.

FIG. 10 is a block diagram of the components of a patient accounting database.

FIG. 11 is an example of profile entries created by a user.

The figures depict one embodiment of the present invention for purposes of illustration only. One skilled in the art will readily recognize from the following discussion that alternative embodiments of the structures and methods illustrated herein may be employed without departing from the principles of the invention described herein.

### DETAILED DESCRIPTION OF THE EMBODIMENTS

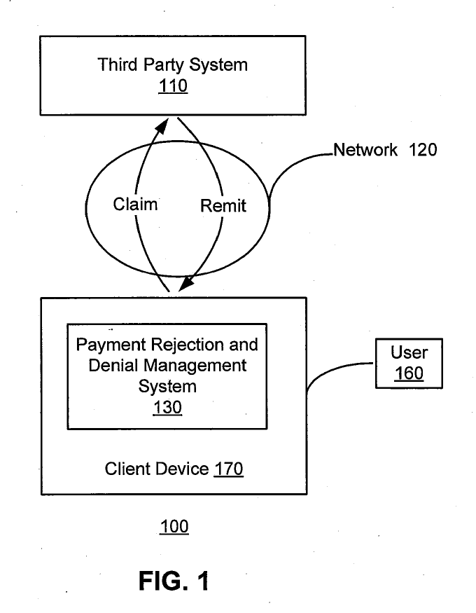

FIG. 1 is a block diagram of the environment in which one embodiment of the present invention operates. Environment 100 includes a payment rejection and denial management system 130 in communication with a third party system 110 over a network 120.

Payment rejection and denial management system 130 is a system utilized by medical service providers, such as hospitals and clinics that submits a claim for rendered medical services to the third party system 110 over communication network 120. System 130 receives from the third party system 110 a remittance advice (hereinafter referred to as “remit”), processes the remit, and in response to the information in the remit performs automatic actions previously defined by a user 160 of system 130. A remit can be a paper document, an electronic document, or any other communication that provides information on how the claim was adjudicated. Communication network 120 can be the hospital network or the Internet.

Third party system 110 represents an entity that pays medical claims (hereinafter referred to as “payer”). For example, the payer 110 can be an employer, insurer, and/or government agency. The payer typically has a policy with patients that describe the terms under which the payer will pay medical expenses incurred by the patient. In addition, the payer 110 often has contracts with medical service providers describing the amounts that the payer 110 will pay for medical services. Although only one third party system 110 is shown in FIG. 1, a person of ordinary skill in the art would understand that system 130 communicates with any number of third party systems 110. In one embodiment, third party system 110 receives claims for medical services from system 130. In response, third party system calculates payment owed to the provider of medical services under the terms of the payer's policies with the patient and/or provider. The payer 110 sends a remit that provides explanation of how the claim was adjudicated.

System 130 operates in several different modes according to the various embodiments and depending on the conditions and needs of a given healthcare information setting. In one embodiment, system 130 operates as a standalone system. When system 130 is implemented as a standalone system, system 130 is executed on a client device 170. The client device 170 can be a personal computer that includes a processor, an addressable memory, and other conventional features (not illustrated) such as a display, local memory, input/output ports, and a network interface. The client device 170 executes a web browser (not shown) for interpreting HTML or other display instructions in a web page and displaying the content accordingly to a user 160. Because an embodiment of the invention is described in the medical context, a user 160 of system 130 can be a physician, office administrator, and other medical staff member having access to system 130. Although one user 160 is shown in FIG. 1 for simplicity only, a person of ordinary skill in the art would understand that any number of permissible users can access system 130.

In another embodiment, system 130 is a client/server or web-based system executed on a server (not shown in FIG. 1). When system 130 is implemented as a client/server system executed on a server, a user 160 of system 130 accesses system 130 over a communication network, such as the Internet, or any other communication mechanism that is capable of supporting communication between a user 160 and payment rejection and denial management system 130. In yet another embodiment, system 130 is integrated into a third party system or framework (not shown in FIG. 1) or a third party portal.

### Payment Rejection and Denial Management System

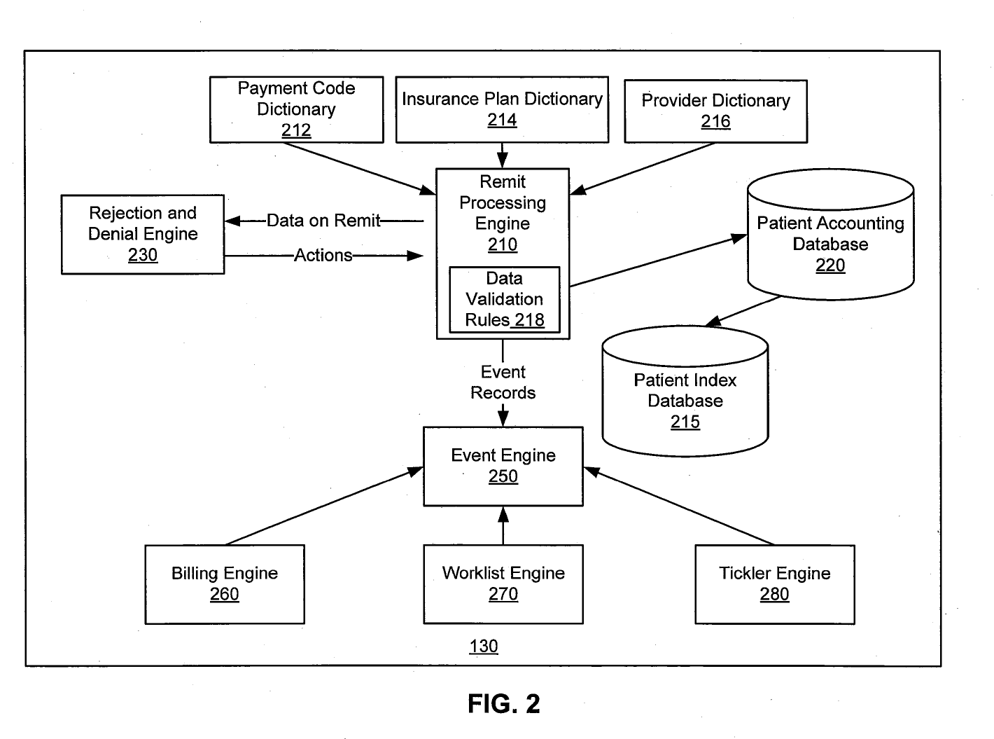

Turning now to FIG. 2, payment rejection and denial management system 130 includes various engines to perform the functionality of the present invention. These engines include a remit processing engine 210, a rejection and denial engine 230, an event engine 250, a billing engine 260, a worklist engine 270, a tickler engine 280, a patient index database 215, a patient accounting database 220, a paycode dictionary 212, an insurance plan dictionary 214, and a provider dictionary 216. In one embodiment, these engines are implemented as modules. As used herein, the term “module” refers to computer program code adapted to provide the functionality attributed to the module. The program code is embodied in a random access memory (RAM), a read-only memory (ROM), or other media.

Remit processing engine 210 is adapted to receive a remit from the third party system 110 and to apply data validation rules 218 to validate the remit. Remit processing engine 210 uses payment code dictionary 212, insurance plan dictionary 214, provider dictionary 216, and patient accounting database 220 to perform data validation process. Payment code dictionary 212 stores valid payment codes used by remit processing engine 210 to account for cash payments on the remit in the patient account database 220. Insurance plan dictionary 214 stores valid insurance plans used by remit processing engine 210 to validate insurance plan information on the remit. Provider dictionary 216 stores valid provider information utilized by remit processing engine 210 to validate provider information on the remit.

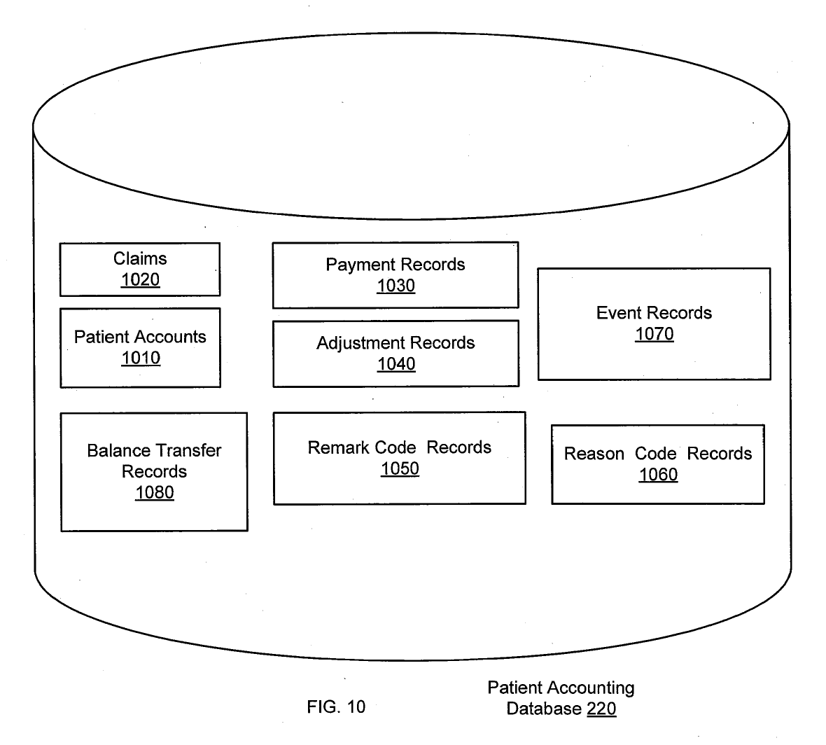

Remit processing engine 210 is adapted to process the data on the remit and to selectively output values on the remit to rejection and denial engine 230. Remit processing engine 210 is further adapted to create various records in the patient accounting database 220. As will be described in more detail in reference to FIG. 10, in one embodiment, remit processing engine 210 creates payment records 1030, adjustment records 1040, remark code records 1050, and reason code records 1060.

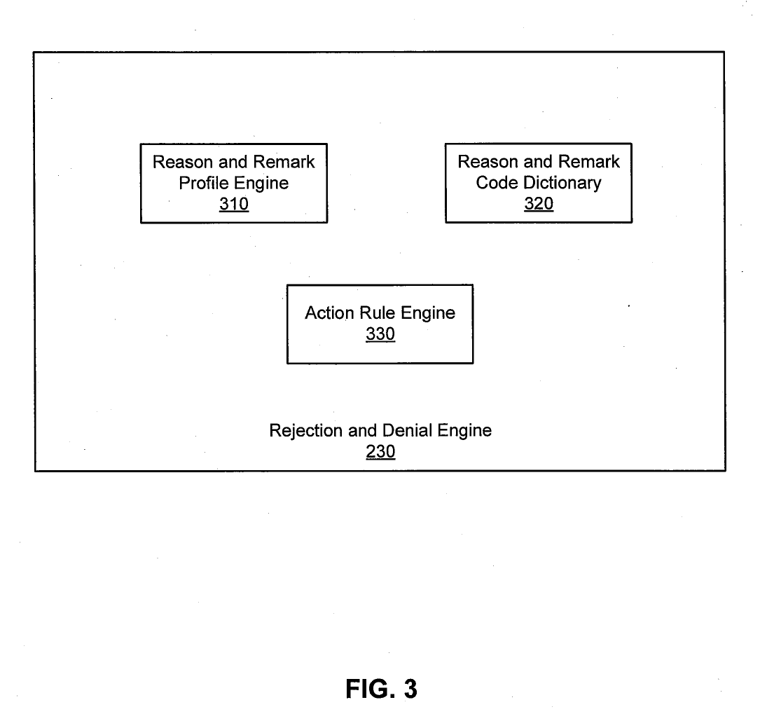

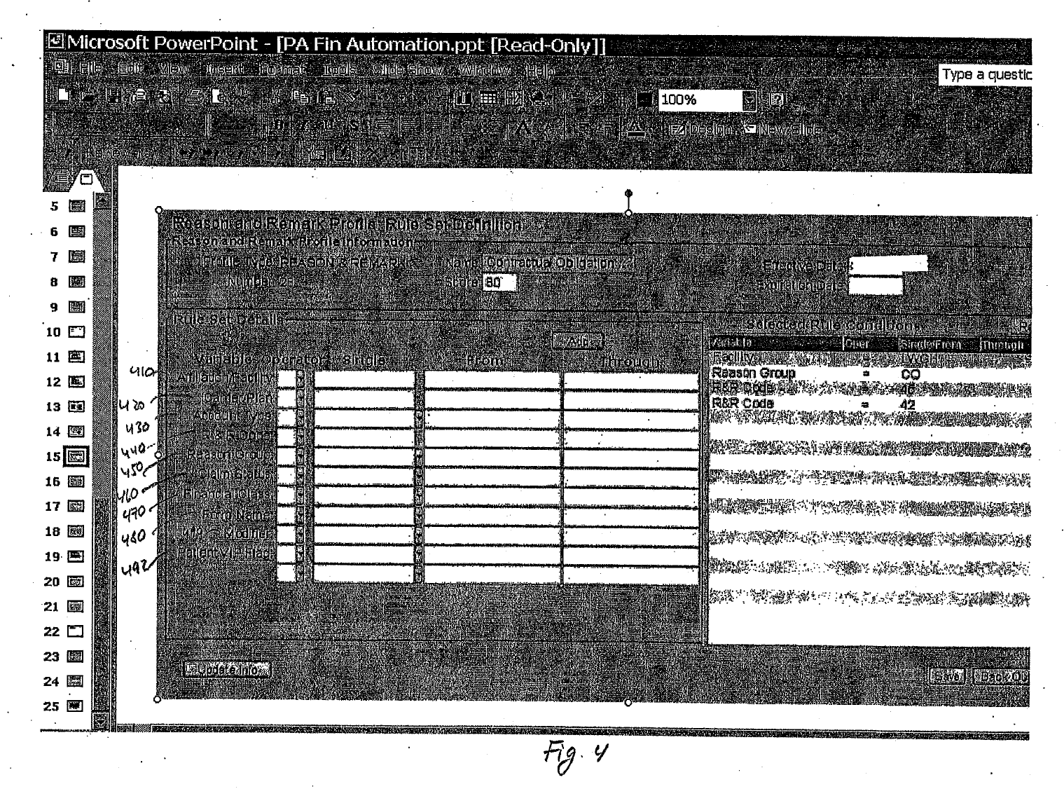

Rejection and denial engine 230 executes a reason and remark profile engine 310 (shown in FIG. 3), which stores profile entries created by a user 160. Each profile entry, in turn, includes a rule set that comprises a group of payment attributes and their associated values, such as the actions to be performed. Exemplary attributes of a rule set will be described in greater detail in reference to FIG. 4. A person of ordinary skill in the art would understand that a user 160 may include any combination of the attributes to create profile entries. A user 160 may designate values/actions for the selected attributes.

Rejection and denial engine 230 further executes an action rule engine 330. Action rule engine 330 receives values from remit processing engine 210 and identifies one or more profile entries that match data on the remit on the greatest number of attributes. Action rule engine 330 identifies one or more actions associated with the selected profile entries. Engine 330 creates one or more adjustment records for adjustment actions and creates one or more event records for non-adjustment types of actions.

Remit processing engine 210 is further adapted to receive actions provided by rejection and denial engine 230. Engine 210 executes adjustment actions.

Rejection and denial engine 230 also executes a reason and remark code dictionary 320 that stores various industry-accepted reason and remark (R&R) codes in compliance with the Health Insurance Portability and Accountability Act (HIPAA).

Referring again to FIG. 2, patient index database 215 stores patient data in the form of patient records. A patient record contains fields for storing data associated with a patient. A field can hold data in the form of numeric, textual, binary information, and any other data type adapted for storage in patient index database 215. In one embodiment, a patient record includes patient identification information, such as a patient ID, patient last name and first name, Social Security Number (SSN), date of birth, gender, and medical record number (MRN).

Event engine 250 is adapted to maintain event records generated by remit processing engine 210 for different types of subsequent processes.

Billing engine 260 is adapted to listen for active billing events, to retrieve a billing event and event status (e.g., active, completed, or pending), and to execute one or more actions in response to active billing events. An exemplary action executed by billing engine 260 can be generating a bill to a payer or holding a bill.

Worklist engine 270 is adapted to listen for active worklist events, to retrieve a worklist event, an event status (active, completed, or pending), and an action attribute, and to execute one or more actions in response to worklist events. An exemplary action performed by worklist engine 270 is, for example, “create a worklist item.”

Tickler engine 280 is adapted to listen for active tickler events, to retrieve a tickler event type, an event status, and an action attribute, and to execute one or more actions in response to tickler events. An exemplary action performed by tickler engine 280 is generating a patient letter.

Patient accounting database 220 stores various records. Referring now to FIG. 10, it features various records stored in patient accounting database. These are patient accounts 1010, claims 1020, payment records 1030, adjustment records 1040, remark code records 1050, event records 1070, reason code records 1060, and balance transfer records 1080.

Patient accounts 1010 are records created for an episode of care performed on a patient. An episode can include one or more services performed on a patient. The record includes a type of service (e.g., inpatient or outpatient), a medical service provider (e.g., a physician's name), and an insurance plan. There maybe more than one patient account records associated with a patient.

Claims 1020 include claims for a particular medical service performed on a patient. Each claim is associated with a patient account record. Claims 1020 can be characterized by a type. For example, a “UB” type indicates that the claim is generated based on the use of a facility. A “HCFA” type indicates that a claim is generated based on professional services (e.g., services performed by a medical professional).

Payment records 1030 are associated with a claim for which the payment was made. A typical payment record includes the amount paid, a payment code, and a payer.

Adjustment records 1040 store information regarding adjustments made in response to the claim. Adjustment records 1040 are associated with a claim for which the adjustment was made. A typical adjustment record indicates a total amount adjusted on a claim, an adjustment code, and a payer.

Reason code records 1060 provide information on how third party system 110 adjudicated a claim. A reason code is associated with a reason amount. An exemplary reason code is “1”. A reason text associated with this reason code is “Deductible Amount” and a reason amount is, for example, $100. A user 160 may elect to set up an automatic action specific to the reason code. For example, a user 160 may elect to move a certain dollar amount with a reason code “1” to self-pay, bypassing secondary or tertiary payers.

Balance transfer records 1080 store information regarding amounts remaining that another payer is responsible to pay. Balance transfer records 1080 are created in pairs reducing accounts receivable balance by the same amount increasing accounts receivable balance. A typical balance transfer record indicates a total amount and a payer.

Remark code records 1050 provide supplemental explanation for an adjustment already described in an adjustment record. A typical remark code record includes a remark code and payer information. A remark code provides additional information on how third party system 110 adjudicated the claim. For example, remark code “M26” indicates that the level of care is not substantiated in the record. A remark code can be an alphanumeric code.

A reason group code provides further information on what can be done with a reason amount. Exemplary reason group codes are “CO” (contractual obligation), “PR” (patient responsibility), and “OA” (other adjustments).

### Exemplary Methods of Operation

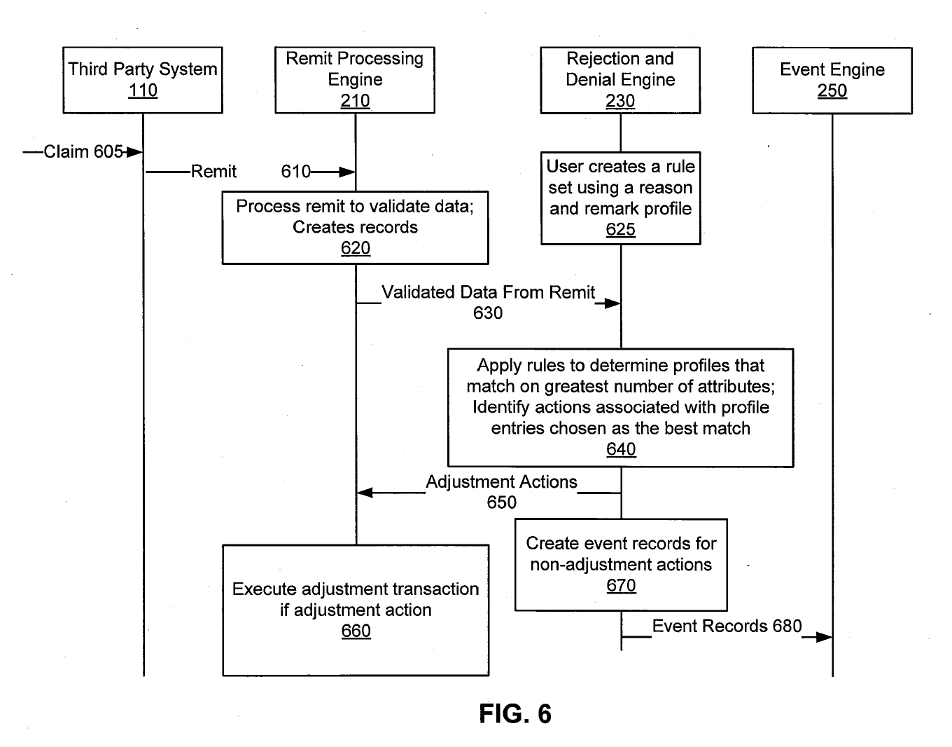

FIG. 6 is an event diagram illustrating exemplary transactions among third party system 110, remit processing engine 210, rejection and denial engine 230, and event engine 250. The horizontal arrows between the vertical lines represent transactions between the associated entities. It should be noted that not every transaction is shown in FIG. 6. In other embodiments of the present invention, the order of the transactions can vary.

A user 160 uses reason and remark profile engine 310 to create 625 profile entries. Each profile entry includes a rule set and one or more associated actions. The rule set comprises of one or more attributes selected by a user. An exemplary list of attributes that a user can select from to create a rule set is shown in FIG. 4. These attributes are: affiliation/facility 410, carrier/plan 420, account type 430, Reason and Remark (R&R) code 440, Reason Group 450, claim status 460, financial class 470, form name 480, modifier 490, and patient VIP flag 492. A user may select any combination of the attributes to create a rule set and to designate values (actions) for the selected attributes. A person of ordinary skill in the art would understand that the list of attributes shown in FIG. 4 is not exhaustive and that system 130 supports any number of attributes.

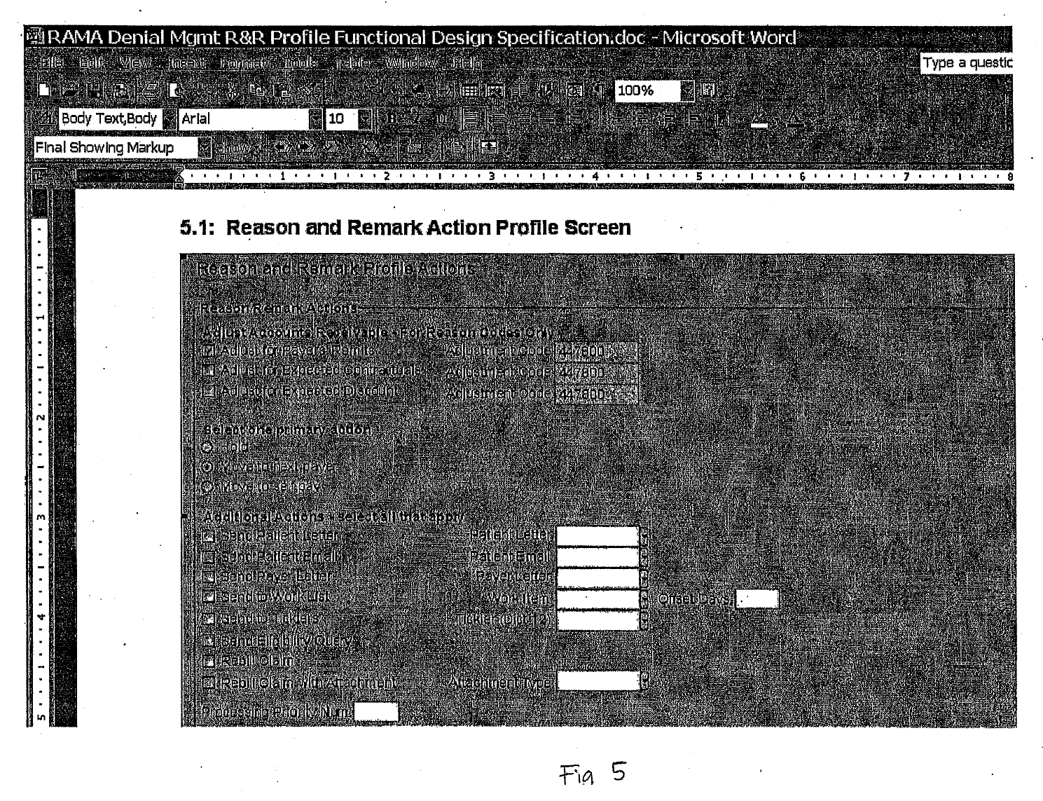

FIG. 4 also features an exemplary rule set created by a user 160. The user designated “LWGH” for facility, “CO” for a reason group, “45” and “42” for R&R code. When creating profile entries, a user may designate one or more actions associated with a particular rule set in the profile. Referring now to FIG. 5, it features various automatic actions that a user can choose from to create a profile entry. As shown in FIG. 5, for the exemplary rule conditions created by a user 160 in FIG. 4, the following actions have been designated: “adjust for payer's remits” and “move to the next payer.”

Thus, the created profile entry represents the following rule conditions:

### IF (Facility=“LWGH” and Reason Group=“CO” and reason code=“42” and “45”)

THEN Adjust for Payer's Remits and Move to the Next Payer.

In addition, a user may assign a score to a profile entry. As will be described below in more detail, the assigned score is used to select one profile entry when more than one profile entry was identified as the best match.

Referring again to FIG. 6, third party system 110 receives a claim 605 for rendered medical services submitted by billing engine 260 (not shown in FIG. 6). Third party system 110 processes the claim and sends 610 a remit. As was previously described, a remit can be a paper document, an electronic document, or any other representation available now or in the future. The remit may include general information about a batch of payment claims processed by third party system 110 as well as specific information related to an individual claim. The general information includes a provider number (a provider can be a hospital, clinic, a physician, or any other facility that renders medical services), the total amount paid on all the claims received over a certain time interval, the total amount adjusted, the total amount processed, insurance plan information, bank account information, and other data relevant for claim processing. The specific account information includes, for example:
an account number
a patient name
a plan subscriber number
a plan group number
a provider
total charges on the claim
allowable amount
amount paid
amount adjusted with group code and readon code
additional reason/remark code information, such as reason/remark group code
claim status (such as processed as primary, processed as secondary, or processed as tertiary)
bill type (inpatient or outpatient)
claim type.

Remit processing engine 210 receives the remit and processes 620 the remit to validate data on the remit. Remit processing engine 210 uses data validation rules 218 to process data on the remit, checking the data against the rules in order. Data validation rules 218 include general data validation rules and specific data validation rules. Exemplary data validation rules 218 of a general category may require that the provider's name, the plan, and the claim number be valid on the remit. Exemplary data validation rules 218 of an account specific category may require that the plan on the remit match the plan on the patient account stored in patient database 220, the account is valid, and the patient account is not in bad debt (e.g., between phases 2 and 7).

In one embodiment, remit processing engine 210 first validates general information on the remit, such as a provider, a claim number, and a plan. To this end, remit processing engine 210 uses claims 1020, insurance plan dictionary 214, and provider dictionary 216. For example, if the plan information is inaccurate, remit processing engine 210 indicates that an error needs to be corrected. After the general information on the remit has been validated, remit processing engine 210 processes specific account information, such as an account number. If the account number is not between phases 2 through 7, it shows that the account is in bad debt. An indication is provided that the account has been sent to a collection agency and the payment received cannot be processed without further intervention.

After remit processing engine 210 validates data on the remit, engine 210 posts the payment, generates various records, and stores the records in patient accounting database 220. In one implementation, remit processing engine 210 generates the following records: payment records 1030, adjustment records 1040, reason code records 1060, and reason remark records 1050 (as was described earlier in reference to FIG. 10).

Remit processing engine 210 processes the validated data on the remit and outputs values for selected attributes. At step 630, remit processing engine 210 makes a server call to rejection and denial engine 230. The server call includes values for the selected attributes in the form of a data set. Rejection and denial engine 230 receives the incoming data set and identifies one or more profile entries that match at least one attribute in the incoming data set. In one implementation, once one or more matching profile entries are identifies, rejection and denial engine 230 identifies 640 the best match. The best match is one or more profile entry that matches the incoming data set on the greatest number of attributes. If more than one profile entry is identified as the best match, rejection and denial engine 230 picks the entry with the highest score.

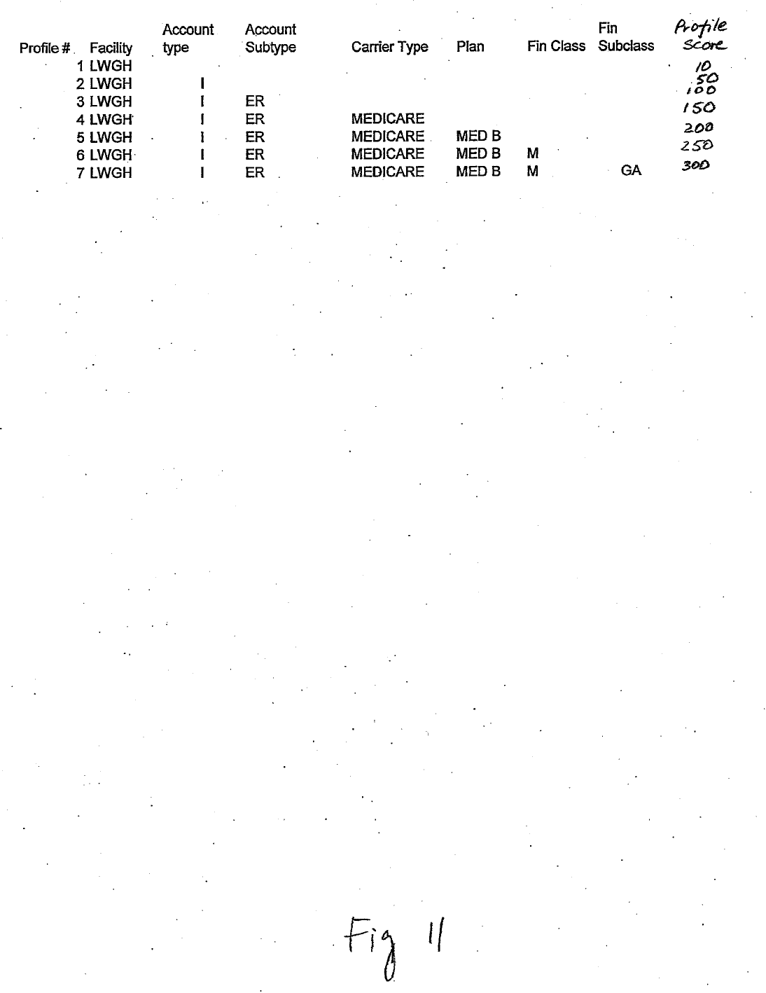

Referring now to FIG. 11, it features exemplary profile entries ##1 through 7 created by a user during the profile set up. Exemplary profile entries shown in FIG. 11 include the following attributes: facility, account type, account subtype, carrier type, plan, financial class, and financial subclass. A person of ordinary skill in the art would understand that a user can create any number of profile entries during the profile set up. By way of an example, a user 160 created the following rule set for the profile entry #4: “LWGH” as a facility, “I” as an account type, and “ER” as an account subtype. Attributes “carrier type”, “plan”, “financial class”, and “financial subclass” do not have values associated with them. A user may assign a score to a profile entry based on various criteria. A user may assign a score based on a number of attributes in a profile entry. A user may assign a score based on an importance of a particular attribute in the profile entry. According to one embodiment, system 130 assigns a higher score to a profile entry that includes values for more specific attributes. For example, in FIG. 11, profile entry #1 has the lowest score among all the profile entries because it includes a value only for the most general attribute (e.g., facility). Profile entry #7 has the highest score because it includes values for the least general attribute, such as a financial subclass. Similarly, the score for profile entry #4 is higher than that for the profile entry #3 because it includes a value for the carrier type, which is more specific than facility, account type, and account subtype.

The following example illustrates how profiles entries are selected. For example, the incoming data on the remit includes the following values: “LWGH” as facility, “I” as account type, and “M” as financial class. In one implementation, rejection and denial engine 230 searches for one or more profile entries that match at least one attribute in the incoming data set. In this example, profiles #1 through #7 match the incoming data set on at least one attribute, “LWGH.” Once one or more matching profile entries are identified, rejection and denial engine 230 identifies one or more profile entries that match on the greatest number of attributes. For example, profile entries #6 and #7 will be selected because they match on all the attributes in the incoming data set. These entries are considered to be the best match.

Since more than two entries have been identified as the best match, rejection and denial engine 230 uses a score associated with the selected entries to identify a profile entry with the highest score. In this example, the score of profile entry #7 is “300.” Profile entry #6 has score “250.” Since the score of profile entry #7 is higher than that of entry #6, profile entry #7 is selected as the best match.

Referring again to FIG. 6, once the best match has been identified, rejection and denial engine 230 identifies one or more actions associated with the identified profile entry. An associated action can be a financial adjustment action, such as for example, “adjust for payer's remit”, “adjust for expected contractuals”, or “adjust for expected discount.” An associated action can be a non-adjustment action, such as “send patient letter”, “send patient email”, “send payer letter”, “send to worklist”, as well as other actions.

If the action is a financial adjustment action, rejection and denial engine 230 communicates 650 the adjustment action to the remit processing engine 210. Remit processing engine 210 executes 660 adjustment actions. If the received action is, for example, to adjust accounts receivables, remit processing engine 210 executes 660 the adjustment transaction using the adjustment code stored in the actions portion of profile entries. For example, as shown in FIG. 5, if the adjustment action is “Adjust For Payers'Remits,” remit processing engine 210 executes the action using adjustment code “447800.”

For non-adjustment actions, rejection and denial engine 230 generates one or more event records with a non-adjustment action and forwards the records to event engine 250. The generated event record may include a patient account number, an attached claim, an event type, and a status (e.g., active, pending, or completed). Exemplary event types supported by system 130 are a billing event, a tickler event, and a worklist event. A person of ordinary skill in the art would understand that this list of events is not exhaustive and that other events are supported by system 130.

Worklist events can be of a guarantor subtype and an insurance subtype. Table 2 provides an exemplary list of worklist events:

TABLE 2
List of Worklist Events

| Guarantor Type | Insurance Type |
|---|---|
| Call the patient | Call the patient |
| Call the payer | Call the payer |
| Create a patient on-demand | Send a claim status |
| letter | request |
| Create a payer on-demand | Send a medical record |
| letter | documentation |
| Evaluate for bad debt write off | Nursing evaluation for |
| | medical necessity denial |
| | Send an eligibility |
| | request |

A billing-type event may include, for example, the following actions: rebill the claim and/or rebill the claim with attachment.

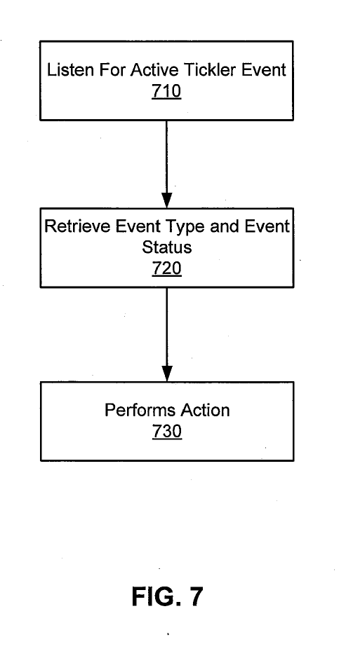

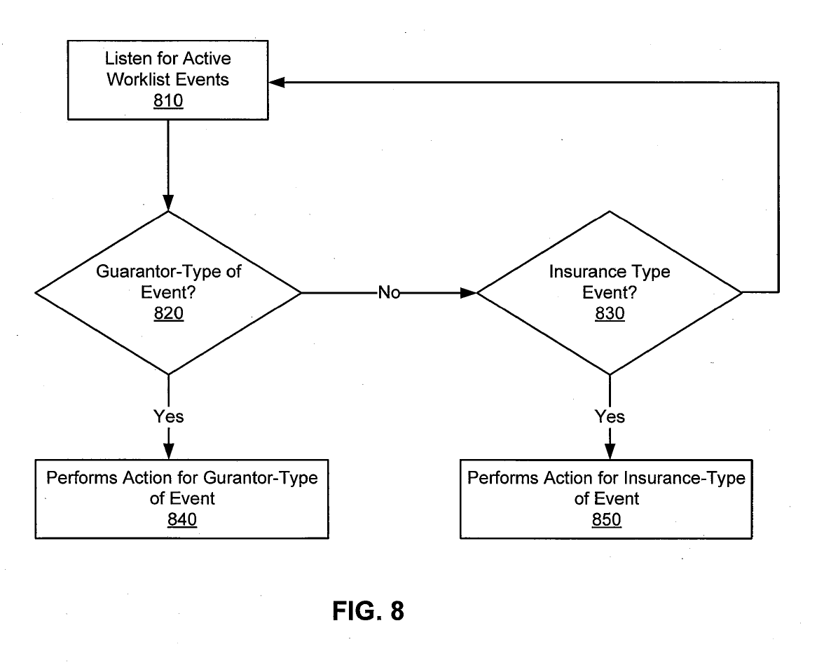

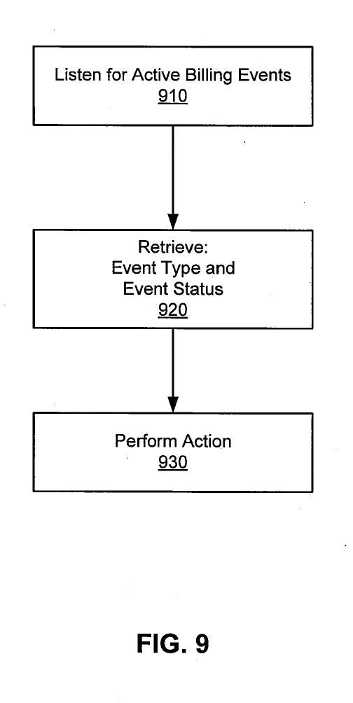

A flow diagram of a method performed by tickler engine 280 is shown in FIG. 7. A flow diagram of a method performed by worklist engine 270 is shown in FIG. 8. A flow diagram of a method performed by billing engine 260 is shown in FIG. 9.

In FIG. 7, initially at step 710, tickler engine 280 listens for active tickler events. A tickler event is triggered when event manager 250 receives an event record with a tickler event type. At step 720, tickler engine 280 identifies the event type and the event status from the event record and executes 730 the tickler event action. Tickler engine 280 updates the tickler event record to mark the event as completed.

Referring now to FIG. 8, at step 810, worklist engine 270 listens for active worklist events. If the event is a guarantor-type of event, worklist engine 270 performs 840 one or more actions associated with the guarantor-type of event. Exemplary actions for the guarantor-type worklist events are shown in Table 2.

If the event is an insurance-type worklist event, worklist engine 270 performs 850 one or more actions associated with the insurance-type worklist event. Exemplary actions for the insurance-type worklist events are shown in Table 2.

Turning now to FIG. 9, a flow chart of a method performed by billing engine 260 is shown. At step 910, billing engine 260 listens for active billing events. Billing engine 260 retrieves 920 a billing event type and an event status and performs 930 an action in response to the billing event. Billing engine 260, for example, may rebill the claim or hold the claim. Upon completion of the action, billing engine 260 marks the event as completed.

In another embodiment, system 130 groups together all codes that were entered for a given claim for a particular day and processes them together. When the incoming data set arrives, rejection and denial engine 230 searches for one or more profile entries that match on the group of codes in the incoming data, rather than matching on the individual code. In this embodiment, a user can create profile entries that can be executed with various permutations.

Thus, the present invention advantageously allows a user to create automatic rules for processing data received from payers. The present invention also allows a user of the system to designate automatic actions. The system executes the actions in response to the data received from the payers. As a result, claims rejections and denials are handled more efficiently, thereby allowing medical service providers to more rapidly seek payments from secondary payers or the patients themselves.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A method for managing payment rejections and denials for claims for rendered medical services, the method comprising:
maintaining a plurality of profile entries created by a user, each profile entry includes a rule set and at least one action associated with the profile entry;
submitting a claim for the rendered medical services to a third party system;
receiving, from the third party system, a communication indicating how the claim for the medical services was adjudicated;
processing the received communication to identify values corresponding to selected attributes;
identifying a profile entry that matches the received values on a greatest number of attributes;
identifying at least one automatic action corresponding to the identified profile entry; and
executing the at least one identified automatic action.

2. The method of claim 1, further comprising using data validation rules to validate values in the received communication.

3. The method of claim 1, wherein the step of identifying profile entries that match the received values on the greatest number of attributes further comprises identifying a profile entry with the highest score.

4. The method of claim 1, further comprising:
responsive to the action being a tickler action, executing the tickler action.

5. The method of claim 1, further comprising:
responsive to the action being a worklist action, executing the worklist action.

6. The method of claim 1, further comprising:
responsive to the action being a billing action, executing the billing action.

7. The method of claim 1, further comprising:
responsive to an action being a non-adjustment action, generating at least one event record.

8. The method of claim 1, further comprising:
responsive to an action being an adjustment action, executing the adjustment action.

9. The method of claim 4, wherein the worklist action comprises at least one action selected from the group comprising:
send a patient letter;
send a patient email;
send a payer letter; and
send to a worklist.

10. The method of claim 8, wherein the billing action comprises at least one action selected from the group comprising:
send an eligibility query;
rebill the claim; and
rebill the claim with and attachement.

11. A system for managing payment rejections and denials for claims for rendered medical services, the system comprising:
a profile engine adapted to maintain a plurality of profile entries, each profile entry includes a rule set and at least one action associated with the profile entry;
a remit processing engine adapted to submit a claim for the rendered medical services to a third party system, to receive, from the third party system, a communication indicating how the claim for the medical services was adjudicated, and to process the received communication to identify values for selected attributes; and
a rejection and denial engine adapted to identify a profile entry that matches the values identified by the remit processing engine on a greatest number of attributes, to identify at least one automatic action corresponding to the identified profile entry, and to execute the at least one identified automatic action.

12. The system of claim 11, wherein the remit processing engine is further adapted to perform an adjustment transaction responsive to the automatic action being an adjustment action.

13. The system of claim 11, wherein the remit processing engine is further adapted to generate event records for non-adjustment actions and wherein the system further comprises:
an event engine adapted to maintain the event records generated by the remit processing engine.

14. The system of claim 13, further comprising a billing engine adapted to listen for at least one active billing event and to execute the at least one active billing event.

15. The system of claim 13, further comprising a tickler engine adapted to listen for at least one active tickler event and to execute the at least one active tickler event.

16. The system of claim 13, further comprising a worklist engine adapted to listen for at least one active worklist event and to execute the at least one active worklist event.

17. The system of claim 11, further comprising:
a patient index database adapted to store patient data;
a patient accounting database for storing adjustment records and event records;
a payment code dictionary adapted to store valid payment codes;
an insurance plan dictionary adapted to store a list of valid insurance plans;
a provider dictionary adapted to store a list of valid medical services providers; and
a reason and remark code dictionary adapted to store valid reason and remark codes.

18. A computer program product comprising:
a computer-readable medium having computer program code embodied therein for managing payment rejections and denials for claims for rendered medical services, the computer program code adapted to:
maintain a plurality of profile entries created by a user, each profile entry includes a rule set and at least one action associated with the profile entry;
submit a claim for the rendered medical services to a third party system;
receive, from the third party system, a communication indicating how the claim for the medical services was adjudicated;
process the received communication to identify values corresponding to selected attributes;

19. The computer program product of claim 18, wherein the computer program code is further adapted to use data validation rules to validate values in the received communication.

20. The computer program product of claim 18, wherein the computer program code is further adapted to identify at least one profile entry having the highest score.

21. The computer program product of claim 18, wherein the computer program code is further adapted to execute a tickler action in response to an action being the tickler action.

22. The computer program product of claim 18, wherein the computer program code is further adapted to execute a worklist action, responsive to the action being the worklist action.

23. The computer program product of claim 18, wherein the computer program code is further adapted to execute a billing action, responsive to the action being the billing action.

24. The computer program product of claim 18, wherein the computer program code is further adapted to generate at least one event record, responsive to an action being a non-adjustment action.

25. The computer program product of claim 18, wherein the computer program code is further adapted to execute an adjustment action, responsive to an action being the adjustment action.
